(19) United States
(12) Patent Application Publication
(10) Pub. No.: US 2017/0058288 A1
(43) Pub. Date: Mar. 2, 2017

(54) OSNF-YA7 GENE FOR INCREASING DROUGHT STRESS RESISTANCE OF PLANT AND USE THEREOF

(71) Applicant: SNU R&DB FOUNDATION, Seoul (KR)

(72) Inventors: Ju-Kon KIM, Gangwon-do (KR); Geupil JANG, Seoul (KR); Hyung il KIM, Gyeonggi-do (KR)

(21) Appl. No.: 15/120,259

(22) PCT Filed: Feb. 19, 2014
(86) PCT No.: PCT/KR2014/001351

§ 371 Date: Aug. 19, 2016

(51) Int. Cl. C12N 15/82; C07K 14/415

(52) U.S. Cl. CPC C12N 15/8273; C07K 14/415

(57) ABSTRACT

A method for enhancing resistance of a plant to abiotic stress includes transforming a plant cell with a recombinant vector which contains a gene encoding NF-YA7 (nuclear factor Y, subunit A7) protein derived from rice (Oryza sativa), and the gene encoding NF-YA7 protein derived from rice of the present invention can be advantageously used for development of a transgenic plant having enhanced resistance to drought resistance.

### TECHNICAL FIELD

The present invention relates to OsNF-YA7 gene for enhancing resistance of a plant to drought stress and a use thereof. More specifically, the present invention relates to a method for enhancing resistance of a plant to drought stress comprising transforming a plant cell with a recombinant vector which includes a gene for encoding NF-YA7 (nuclear factor Y, subunit A7) protein derived from rice (Oryza sativa), a method for producing a transgenic plant having enhanced resistance to abiotic stress compared to the wild type by transforming a plant cell with a recombinant vector which includes the gene for encoding NF-YA7 protein, a transgenic plant produced by the aforementioned method and a seed thereof, and a composition for enhancing resistance of a plant to abiotic stress which contains the gene for encoding NF-YA7 protein as an effective component.

### BACKGROUND ART

Drought stress is one of the factors which present the most serious challenges to crop production worldwide. Upon exposure of a plant to drought conditions, many stress-related genes are induced and their products are thought to function as cellular protectants of stress-induced damage. The expression of stress-related genes is largely regulated by specific transcription factors. Members of the AP2, bZIP, zinc finger, and MYB families have been shown to play a transcription regulatory role in stress responses. The rice and Arabidopsis genomes encode more than 1300 transcriptional regulators, accounting for about 6% of the estimated total number of genes in both cases. Also, about 45% of these transcription factors were reported to be from plant-specific families.

Nuclear factor Y (NF-Y) is transcription factor complex which binds specifically to highly-conserved CCAAT sequence of target promoters (Romier et al., 2003, J. Biol. Chem. 278:1336-1345). NF-Y transcription complex is composed of three subunits, NF-YA, NF-YB and NF-YC. Each subunit contains conserved domains that are required for DNA binding and protein interactions. NF-YB and NF-YC initially bind to each other in the cytoplasm to form a NF-YB/NF-YC dimeric complex and then move into the nucleus where heterotrimeric complex is formed by interaction between NF-YA and NF-YB/NF-YC dimeric complex. NF-Y transcription factor exists in all higher eukaryotes including mammals and plants. However, interestingly, number of genes encoding NF-Y transcription factor are very different between mammals and plants. In mammals NF-Y transcription factor is encoded by single gene whereas plant NF-Y transcription factor is encoded by gene families in plants. These suggested that multiple copy of NF-Y in plants may cause a regulatory complexity through various interactions between NF-Y subunit members.

NF-YA subunit carries two conserved domains in N-terminal and C-terminal regions domain; N-terminal domain is required for interaction with NF-YB/NF-YC heterodimer, and the C-terminal domain is required for DNA-binding.

Accordingly, the inventors of the present invention isolated two NF-YA transcription factors from rice (Oryza sativa), i.e., NF-YA4 and NF-YA7, and analyzed the function of those genes regarding drought resistance.

Meanwhile, although US Patent Publication No. 2009-0089899 discloses ‘Method for Enhancing Drought Stress Tolerance in Plants by Active AREB1’, OsNF-YA7 gene for enhancing resistance of a plant to drought stress and a use thereof as described in the present invention are not included therein.

### DETAILED DESCRIPTION OF THE INVENTION

### Technical Problems to be Solved

The present invention is devised in view of the above-described needs. The inventors of the present invention prepared a recombinant vector comprising a gene for encoding NF-YA7 protein derived from rice, and, according to transformation of a plant with this recombinant vector, they produced a transgenic rice plant which overexpresses NF-YA7 gene. By confirming that, when treated with drought stress, the transgenic rice plant which overexpresses NF-YA7 gene has enhanced resistance to drought stress compared to the wild type plant, the inventors completed the present invention.

### Technical Means for Solving the Problems

To solve the above problems, provided by the present invention is a method for enhancing resistance of a plant to abiotic stress, comprising transforming a plant cell with a recombinant vector containing a gene encoding NF-YA7 (nuclear factor Y, subunit A7) protein derived from rice (Oryza sativa).

Also provided by the present invention is a method for producing a transgenic plant having enhanced resistance to abiotic stress compared to the wild type, comprising transforming a recombinant vector containing a gene encoding NF-YA7 protein derived from rice.

Also provided by the present invention is a transgenic plant produced by the above method which has enhanced resistance to abiotic stress compared to the wild type, and a seed thereof.

Also provided by the present invention is a composition for enhancing resistance of a plant to abiotic stress which contains, as an effective component, the gene encoding NF-YA7 protein derived from rice.

### Advantageous Effect of the Invention

According to the present invention, the plant which overexpresses the gene encoding NF-YA7 protein derived from rice exhibits enhanced resistance to stress at drought conditions compared to the wild type. As the gene encoding NF-YA7 protein derived from rice of the present invention can be advantageously used for development of a transgenic plant which has enhanced resistance to drought stress, it can be very advantageously used in the industry.

### BRIEF DESCRIPTION OF THE DRAWINGS

FIG. 1 is a drawing illustrating a change in expression level of OsNF-YA 7 and OsNF-YA4 when a rice plant has been subjected to a stress treatment of drought, salt, or low temperature.

FIGS. 2a and 2b are drawings illustrating a change in expression level of OsNF-YA7 and OsNF-YA4 when a plant has been treated with abscisic acid (ABA) as a stress hormone, either for short-term period (FIG. 2a) or long-term period (FIG. 2b).

FIGS. 3a and 3b are drawings illustrating the result of comparing the resistance to drought stress of a transgenic plant which overexpresses OsNF-YA 7 and OsNF-YA4 (i.e., PGD1: OsNF-YA4 and PGD1: OsNF-YA7) to the wild type plant, in which FIG. 3a is a photographic image showing the appearance of a plant after drought stress treatment followed by watering and FIG. 3b is a graph showing the analysis of a change in Fv/Fm value in accordance with the drought stress treatment. The top graph corresponds to the result of a plant transformed with OsNF-YA4 and the bottom graph corresponds to the result of a plant transformed with OsNF-YA 7.

### BEST MODE(S) FOR CARRYING OUT THE INVENTION

To achieve the object of the present invention, the present invention provides a method for enhancing resistance of a plant to abiotic stress, comprising transforming a plant cell with a recombinant vector containing a gene encoding NF-YA7 (nuclear factor Y, subunit A7) protein which is derived from rice (Oryza sativa).

The scope of the NF-YA7 protein according to the present invention includes a protein having an amino acid sequence represented by SEQ ID NO: 2, and also functional equivalents of the protein. The term “functional equivalent” indicates a protein having, as a result of addition, substitution, or deletion of an amino acid, at least 70%, preferably at least 80%, more preferably at least 90%, and even more preferably at least 95% sequence homology with the amino acid sequence represented by SEQ ID NO: 2, and it indicates a protein exhibiting substantially the same physiological activity as the protein represented by SEQ ID NO: 2. The expression “substantially the same physiological activity” means an activity of enhancing resistance of a plant to abiotic stress.

Also provided by the present invention is a gene which encodes the NF-YA7 protein. The gene of the present invention may include the nucleotide sequence which is represented by SEQ ID NO: 1. Further, homologues of the nucleotide sequence are also within the scope of the present invention. Specifically, the above described gene may comprise a nucleotide sequence which has preferably at least 70%, more preferably at least 80%, still more preferably at least 90%, and most preferably at least 95% homology with the nucleotide sequence of SEQ ID NO: 1. The “sequence homology %” for a certain polynucleotide is identified by comparing a comparative region with two sequences that are optimally aligned. In this regard, a part of the polynucleotide in comparative region may comprise an addition or a deletion (i.e., a gap) compared to a reference sequence (without any addition or deletion) relative to the optimized alignment of the two sequences.

The term “recombinant” indicates a cell which replicates a heterogeneous nucleotide or expresses said nucleotide, or a peptide, a heterogeneous peptide, or a protein encoded by a heterogeneous nucleotide. Recombinant cell can express a gene or a gene fragment in the form of a sense or antisense, which are not found in natural state of cell. In addition, a recombinant cell can express a gene that is found in natural state, provided that said gene is modified and re-introduced into the cell by an artificial means.

According to the present invention, the sequence of the gene encoding NF-YA7 protein can be inserted to a recombinant expression vector. The term “recombinant expression vector” means bacteria plasmid, phage, yeast plasmid, plant cell virus, mammalian cell virus, or other vector. Any plasmid and vector can be generally used if it can replicate and is stabilized in a host. Important characteristics of the expression vector include that it comprises a replication origin, a promoter, a marker gene, and a translation control element.

The expression vector comprising the gene sequence of the NF-YA7 protein and an appropriate signal for regulating transcription/translation can be constructed according to a method which is well known to a skilled person in the art. The method includes an in vitro recombinant DNA technique, a DNA synthesis technique, and an in vivo recombinant technique. For inducing mRNA synthesis, the DNA sequence can be effectively linked to a suitable promoter present in the expression vector. In addition, the expression vector may comprise a ribosome binding site as a translation initiation site and a transcription terminator.

Preferred example of the recombinant vector of the present invention is Ti-plasmid vector which can transfer a part of itself, i.e., so called T-region, to a plant cell when the vector is present in an appropriate host such as Agrobacterium tumefaciens. Other types of Ti-plasmid vector (see, EP 0 116 718 B1) are currently used for transferring a hybrid DNA sequence to protoplasts that can produce a new plant by appropriately inserting a plant cell or hybrid DNA to a genome of a plant. Especially preferred form of Ti-plasmid vector is a so-called binary vector which has been disclosed in EP 0 120 516 B1 and U.S. Pat. No. 4,940,838. Other vector that can be used for introducing the DNA of the present invention to a host plant can be selected from a double-stranded plant virus (e.g., CaMV), a single-stranded virus, and a viral vector which can be originated from Gemini virus, etc., for example a non-complete plant viral vector. Use of said vector can be advantageous especially when a host plant cannot be easily transformed.

The recombinant vector may comprise at least one selective marker. Said selective marker is a nucleotide sequence having a property of being selected by a common chemical method. Examples include all genes that are useful for distinguishing transformed cells from non-transformed cells. Specific examples thereof include a gene resistant to herbicide such as glyphosate and phosphinotricine, and a gene resistant to antibiotics such as kanamycin, G418, bleomycin, hygromycin, and chloramphenicol, but not limited thereto.

For the recombinant vector according to the present invention, the promoter may be any of CaMV 35S promoter, actin promoter, ubiquitin promoter, pEMU promoter, MAS promoter, and histone promoter, but not limited thereto. The term “promoter” means a DNA molecule to which RNA polymerase binds in order to initiate its transcription, and it corresponds to a DNA region upstream of a structural gene. The term “plant promoter” indicates a promoter which can initiate transcription in a plant cell. The term “constitutive promoter” indicates a promoter which is active in most of environmental conditions and development states or cell differentiation states. Since a transformant can be selected with various mechanisms at various stages, the constitutive promoter can be preferable for the present invention. Therefore, a possibility for choosing the constitutive promoter is not limited herein.

For the recombinant vector of the present invention, any conventional terminator can be used. Examples include nopaline synthase (NOS), rice α-amylase RAmy1 A terminator, a phaseoline terminator, a terminator for optopine gene of Agrobacterium tumefaciens, or the like, but are not limited thereto. Regarding the necessity of terminator, it is generally known that such region can increase reliability and an efficiency of transcription in plant cells. Therefore, the use of terminator is highly preferable in view of the contexts of the present invention.

Transformation of a plant means any method by which DNA is delivered to a plant. Such transformation method does not necessarily need a period for regeneration and/or tissue culture. Transformation of plant species is now quite general not only for dicot plants but also for monocot plants. In principle, any transformation method can be used for introducing a hybrid DNA of the present invention to appropriate progenitor cells. The method can be appropriately selected from a calcium/polyethylene glycol method for protoplasts (Krens, F. A. et al., 1982, Nature 296, 72-74; Negrutiu I. et al., June 1987, Plant Mol. Biol. 8, 363-373), an electroporation method for protoplasts (Shillito R. D. et al., 1985 Bio. Technol. 3, 1099-1102), a microscopic injection method for plant components (Crossway A. et al., 1986, Mol. Gen. Genet. 202, 179-185), a particle bombardment method for various plant components (DNA or RNA-coated) (Klein T. M. et al., 1987, Nature 327, 70), or a (non-complete) viral infection method in Agrobacterium tumefaciens mediated gene transfer by plant invasion or transformation of fully ripened pollen or microspore (EP 0 301 316), etc. A method preferred in the present invention includes Agrobacterium mediated DNA transfer. In particular, so-called binary vector technique as disclosed in EP 0 120 516 and U.S. Pat. No. 4,940,838 can be preferably adopted for the present invention.

Regarding the production method according to one embodiment of the present invention, the abiotic stress may be drought, oxidation, heat, light, low temperature, or salt stress. Preferably it is drought stress, but not limited thereto.

Also provided by the present invention is a method for producing a transgenic plant having enhanced resistance to abiotic stress compared to the wild type, comprising:

transforming a plant cell with a recombinant vector which contains a gene encoding NF-YA7 protein derived from rice; and

regenerating a plant from the transformed plant cell.

Regarding the production method according to one embodiment of the present invention, the scope of NF-YA7 protein is as defined above.

The method of the present invention comprises a step of transforming a plant cell with the recombinant vector of the present invention, and the transformation may be mediated by Agrobacterium tumefiaciens, for example. Further, the method of the present invention comprises a step of regenerating a transgenic plant from the transformed plant cell. As for the method for regenerating a transgenic plant from a transformed plant cell, a method well known in the pertinent art can be used.

The transformed plant cell needs to be regenerated into a whole plant. Techniques for regenerating a mature plant based on culture of callus or protoplast have been well known in the pertinent art for various species.

Regarding the production method according to one embodiment of the present invention, the abiotic stress may be drought, oxidation, heat, light, low temperature, or salt stress. Preferably it is drought stress, but not limited thereto.

Also provided by the present invention is a transgenic plant produced by the above method which has enhanced resistance to abiotic stress compared to the wild type, and a seed thereof.

With regard to one embodiment of the present invention, the plant can be preferably a monocot plant such as rice, barley, wheat, rye, corn, sugar cane, oat, or onion, or a dicot plant such as potato, eggplant, tobacco, pepper, tomato, burdock, crown daisy, lettuce, balloon flower, spinach, chard, yam, carrot, water parsley, Chinese cabbage, cabbage, Raphanus sativus for. raphnistroides MAK, watermelon, oriental melon, cucumber, zucchini, gourd, strawberry, soybean, mung bean, kidney bean, or sweet pea. More preferably, it can be a monocot plant, and even more preferably rice. However, it is not limited thereto.

Also provided by the present invention is a composition for enhancing resistance of a plant to abiotic stress which contains, as an effective component, a gene for encoding NF-YA7 protein consisting of the amino acid sequence of SEQ ID NO: 2. The composition contains, as an effective component, a gene for encoding NF-YA7 protein consisting of the amino acid sequence of SEQ ID NO: 2, and by transforming a plant with this gene, the resistance of a plant to abiotic stress can be enhanced.

Hereinbelow, the present invention is explained in greater detail in view of the Examples. However, it is evident that the following Examples are given only for exemplification of the present invention and by no means the present invention is limited to the following Examples.

Materials and Methods

Plasmid Construction and Rice Transformation

Total RNA was isolated from rice (breed: “Nakdong”) and the full-length cDNA of OsNF-YA4 (AK069854) and OsNF-YA7 (AK059903) was amplified by RT-PCR (Promega, Madison, Wis.) using the total RNA as a template. The primers used for the amplification include OsNF-YA4 forward primer to (5′-ATGGAGTCGAGGCCGGGGGG-3′; SEQ ID NO: 3) and OsNF-YA4 reverse primer (5′-TCATGTTTCCTTCTGTAGGA-3′; SEQ ID NO: 4), and OsNF-YA7 forward primer (5′-ATGAAGCCAGATGGTGAAAC-3′; SEQ ID NO: 5) and OsNF-YA7 reverse primer (5′-TCATACAACATCGGACGCAT-3′; SEQ ID NO: 6). Each full-length cDNA obtained after amplification was inserted into p700 vector carrying PGD1 promoter using Gateway system (Invitrogen, Carlsbad, Calif.). PGD1: OsNF-YA7 and PGD1: OsNF-YF4 vectors were introduced to a plant by Agrobacterium-mediated co-cultivation method, as described previously (Tang et al., 1999, Mol. Breeding 5: 453-461).

Stress and absicic acid (ABA) treatment

To analyze expression patterns of OsNF-YA4 and OsNF-YA7 gene in response to abiotic stress, non-transgenic rice plants (NT plants; breed—“Nakdong”) were grown in soil for 4 week in green house. The whole plant of a 4-week old non-transgenic plant was air-dried for 3 hrs for drought stress treatment. For the treatment of salt stress, whole plants were transferred to a solution containing 200 mM of NaCl and kept for 24 hours. For the treatment of low temperature stress, whole plants were transferred to a 4° C. low temperature chamber and kept for 24 hrs. For the analysis of OsNF-YA4 and OsNF-YA7 gene expression pattern in response to ABA, NT plant were grown in MS media for 2 week in growth chamber. For the short-term treatment with ABA, the whole plants were transferred to a solution containing 10 or 100 μM ABA and treated with the solution for 24 hours. For the long-term treatment with ABA, NT plant seeds were planted and grown on MS media supplemented with 0, 0.5, 1 or 3 μM of ABA for 2 weeks.

Real-Time Quantitative Reverse Transcription PCR

Leaf and root tissues of a plant which has been treated with either abiotic stress or absicic acid were collected and frozen. Then, the frozen leaf and root tissues were ground in liquid nitrogen and total RNA was extracted from them by using a Qiagen RNeasy plant mini kit (Qiagen, Valencia, Calif.). One microgram of the total RNA was used to synthesize the first-strand cDNA using Superscript II cDNA synthesis system (Invitrogen, USA). To analyze the gene expression level, quantitative PCR was carried out by using Mx3000p real-time PCR machine and Platinum® SYBR® Green qPCR SuperMix-UDG system (Invitrogen, USA). Rice ubiquitin transcript was used as a reference for normalization and primer sequences specific to the OsNF-YA4 and OsNF-YA7 genes were formed at 3′ UTR and 3′ exon regions. The primer used for qRT-PCR is as shown in the following Table 1.

TABLE 1

| | | Nucleotide sequence |
|---|---|---|
| Gene name | | information (5′→3′) |
| OsNF-YA4 | Forward | GGATCCCTCCTGACTTACAG |
| | direction | (SEQ ID NO: 7) |
| | Reverse | GGATTCTTGCTCCAGAATTG |
| | direction | (SEQ ID NO: 8) |
| OsNF-YA7 | Forward | GGAGAACTCGAGCCCAACAA |
| | direction | (SEQ ID NO: 9) |
| | Reverse | GCAAGCACACAATTCGATCGA |
| | direction | (SEQ ID NO: 10) |
| OsUbi. | Forward | ATGGAGCTGCTGCTGTTCTA |
| | direction | (SEQ ID NO: 11) |
| | Reverse | TTCTTCCATGCTGCTCTACC |
| | direction | (SEQ ID NO: 12) |

Phenotypical analysis for drought resistance

PGD1: OsNF-YA4 and PGD1: OsNF-YA7 transgenic plants were grown in soil for 4 weeks together with NT plants. Drought stress was applied by removing water from the soils. 7 Days after the drought treatment, re-watering was performed. Thereafter, drought-induced visual symptoms were observed by capturing images of—PGD1: OsNF-YA4 and PGD1: OsNF-YA7 transgenic plants and NT plant.

PAM (Pulse-Amplitude Modulation) Test

Three lines of PGD1: OsNF-YA4 and PGD1: OsNF-YA7 transgenic plants were grown in soil for 2 weeks together with NT plants. The leaves were collected from each plant and then adapted in dark condition for 10 minutes. For PAM analysis, drought condition was applied with time-course manners in growth chamber. At each indicted time, Fv/Fm values were measured with a pulse modulation fluorometer (min-PAM, Walz, Germany) by detecting chlorophyll fluorescence emitted from the upper surface of the leaves. Fv/Fm values indicate the activity of Photosystem II.

### EXAMPLE 1

### Expression Analysis of OsNF-YA4 and OsNF-YA7 Genes in Abiotic Stress Conditions

Based on previous results of microarray, it has been confirmed that expression of two OsNF-YA genes (OsNF-YA4 and OsNF-YA7) are induced by drought stress. To confirm a change in the expression pattern of OsNF-YA4 and OsNF-YA7 genes in response to abiotic stress including drought stress, the NT rice was exposed to drought, high salt, or low temperature stress.

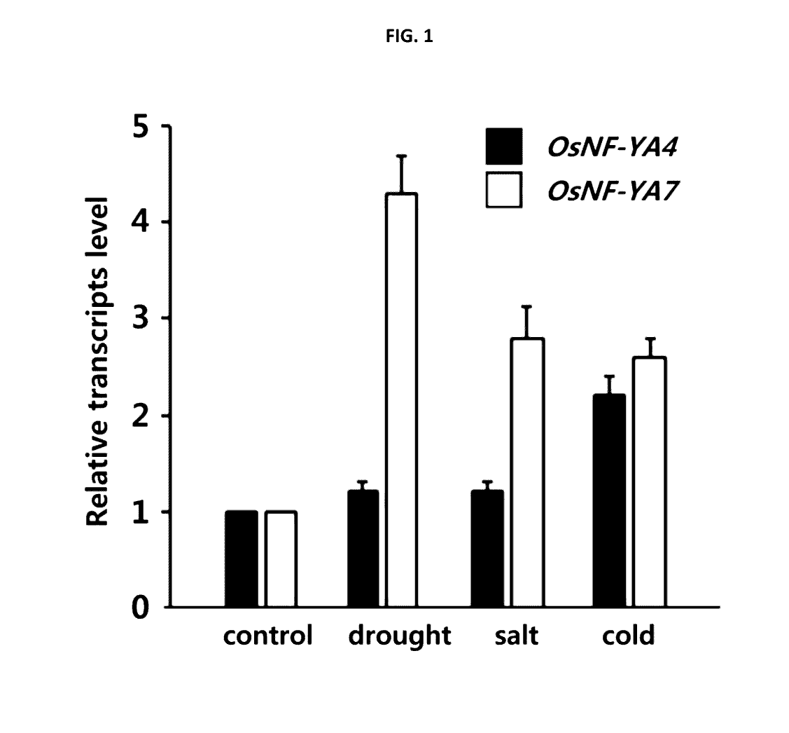

As a result, it was found that, compared to the control group without stress treatment, OsNF-YA7 gene expression amount was increased under stress conditions; i.e., 4.4 fold increase by drought stress, 2.9 fold increase by high salt stress and 2.7 fold increase by low temperature stress (FIG. 1). On the other hand, compared to the control group, the expression amount of OsNF-YA4 gene has increased only by low temperature stress treatment, while there was almost no change in the expression amount by drought stress treatment or high salt stress treatment.

### EXAMPLE 2

### Analysis of Expression Pattern of OsNF-YA4 and OsNF-YA7 Genes in Response to ABA Treatment

Abscisic acid (ABA) is one of plant hormones for regulating plant growth and development and it is an important substance for regulating the response and adaptation of a plant to abiotic stress. Its content particularly increases under the abiotic stress conditions such as cold temperature, drought, or salt., and when a plant is treated with absicic acid, a situation similar to a case under stress conditions can be induced. To examine the interaction between ABA and these OsNF-YA genes, a change in expression level of OsNF-YA4 and OsNF-YA7 was analyzed after treating a NT rice plant with absicic acid.

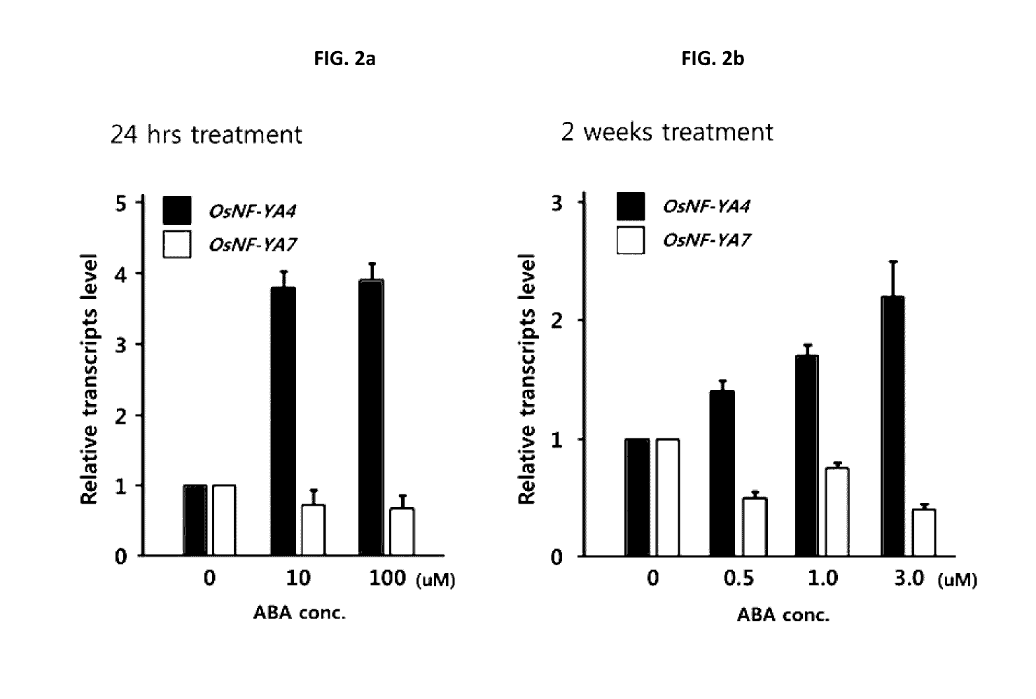

As a result, the short-term treatment with 10 and 100 μM ABA led to approximately 30% decrease in OsNF-YA7 expression level while 4-fold higher increase was shown in OsNF-YA4 expression level compared to untreated control group (FIG. 2a). The long-term treatment with ABA also showed a similar result (FIG. 2b). These results indicate that expression of OsNF-YA4 gene and OsNF-YA7 gene is differently regulated under stress conditions caused by absicic acid.

### EXAMPLE 3

### Determination of Drought Resistance Based on Overexpression of OsNF-YA7 Gene

From the above Example 1, it was confirmed that OsNF-YA7 gene expression is highly induced under drought stress conditions. Accordingly, to confirm the potential function of OsNF-YA7 gene relating to drought resistance, a transgenic plant overexpressing OsNF-YA4 and OsNF-YA7 was subjected to a drought stress treatment.

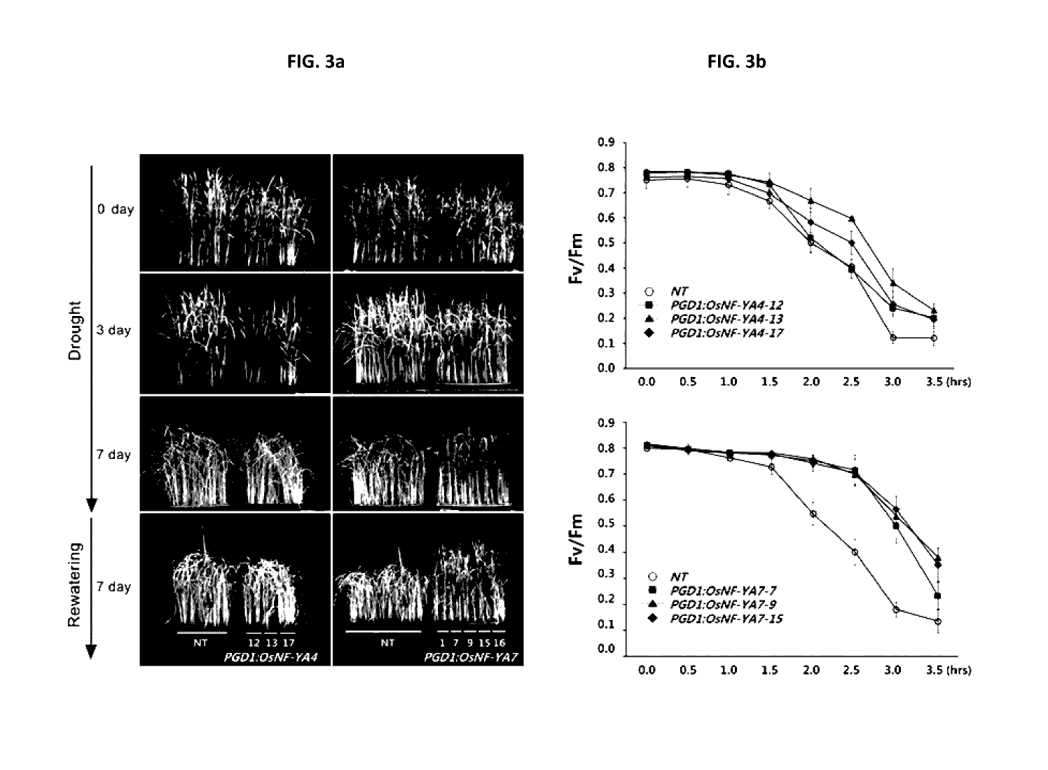

3 Days after the drought treatment, NT plants displayed visual symptoms of slight drought-induced damages such as leaf rolling and wilting. With increase of exposure time to drought stress treatment, the symptoms became severe in the NT plants, and, on day 7 of the drought treatment, significant damages to NT plants were shown. All the leaves of the NT plants were dried out and re-watering was not enough to have the NT plants recovered from the drought-induced damages (FIG. 3a). Similar results to the NT plants were obtained also from the OsNF-YA4-overexpressing transgenic plants, i.e., with increase of exposure time to drought stress treatment, the drought stress-induced symptoms became severe in the OsNF-YA4-overexpressing transgenic plants and re-watering was not enough to have them recovered from the drought-induced damages. However, OsNF-YA7-overexpressing transgenic plants displayed strong resistance against drought stress. Namely, even on day 3 after the drought stress treatment, the OsNF-YA 7-overexpressing transgenic plants exhibited almost the same state as the non-treated control group. 7 Days after the drought treatment, there was a symptom of damages that are caused by the stress, but the degree of the symptom was found to be lower than the NT plant which has been treated with drought stress. Furthermore, re-watering was able to have the plant recovered from the drought stress-induced damages. These results indicate that the OsNF-YA7 gene can enhance the resistance to drought stress.

To confirm the function of OsNF-YA7 in drought resistance, PAM (Pulse-Amplitude Modulation) test was carried out by using 3 independent lines of OsNF-YA7-overexpressing transgenic plant. The Fv/Fm value of PAM analysis is used for monitoring the effect of abiotic stress on the plant. The drought stress treatment strongly lowered the Fv/Fm value in NT plants, i.e., Fv/Fm value of NT plants was 0.8 at no stress treatment conditions, but 1 hour after the drought stress treatment, a decrease in Fv/Fm value was shown, eventually exhibiting a result that the decrease in Fv/Fm values is accelerated with an increase of exposure time to the stress (FIG. 3b, NT). In contrast to NT plants, the decrease in Fv/Fm values by drought stress was delayed in OsNF-YA 7-overexpressing plants. Three OsNF-YA 7-overexpressing transgenic plants showed Fv/Fm values that are similar to those of non-treatment control group until 2.5 hrs after the drought stress treatment. After 3 hours, however, the results showing a decrease in the Fv/Fm value were yielded (FIG. 3b).

However, the OsNF-YA4-overexpressing transgenic plant did not show, after the drought stress treatment, a tendency of having delayed decrease in the Fv/Fm as it has been confirmed from the OsNF-YA7-overexpressing transgenic plant. The OsNF-YA4-overexpressing transgenic plant showed a result having a difference in the Fv/Fm value which is very small or almost negligible compared to the delayed decrease in the Fv/Fm value of the NT plants. These results support that overexpression of OsNF-YA7 can enhance the resistance of a plant against drought.

1. A method for enhancing resistance of a plant to abiotic stress, comprising transforming a plant cell with a recombinant vector which contains a gene encoding NF-YA7 (nuclear factor Y, subunit A7) protein derived from rice (Oryza sativa).

2. The method for enhancing resistance of a plant to abiotic stress according to claim 1, wherein the NF-YA7 protein consists of an amino acid sequence represented by SEQ ID NO: 2.

3. The method for enhancing resistance of a plant to abiotic stress according to claim 1, wherein the abiotic stress is a drought stress.

4. A method for producing a transgenic plant having enhanced resistance to abiotic stress compared to a wild type, comprising:
transforming a plant cell with a recombinant vector which contains a gene encoding NF-YA7 (nuclear factor Y, subunit A7) protein derived from rice; and
regenerating a plant from the transformed plant cell.

5. The method according to claim 4, wherein the NF-YA7 protein consists of an amino acid sequence represented by SEQ ID NO: 2.

6. The method according to claim 4, wherein the abiotic stress is a drought stress.

7. A transgenic plant produced by the method according to claim 4 which has enhanced resistance to abiotic stress compared to a wild type.

8. The plant according to claim 7, wherein the abiotic stress is a drought stress.

9. The plant according to claim 7, wherein the plant is a monocot plant.

10. A seed of the plant according to claim 7.

11. A composition for enhancing resistance of a plant to abiotic stress which contains, as an effective component, a gene for encoding NF-YA7 (nuclear factor Y, subunit A7) protein consisting of the amino acid sequence of SEQ ID NO: 2.


SEQUENCE LISTING (12 sequences)

SEQ ID NO: 1 - DNA, 582 bp, Oryza sativa
atgaagccagatggtgaaactcagcttcgtcctacagctgctggacatccagatcctggtttgggcacatcatctgcagaatacgtggcctctctaggaccagctacagctccagtatcttatccatatatcagtacctattatggaggcacttatggtgcttatagtggacaacctctggttaacgctgctttaatggcaatgcctccgcattctgtgcccttggtgaccgatgctgttgtagagcccatatatgtcaatgcaaggcagtatcatggtatattaaggcggcgccaatctcgtgcaaaagctgaatcagaaaataaggccaacaaaatccgcaagccttacctacatgagtcccgccatctgcatgctttgaaaagggcaagggggtccggtggtcgatttctcaactctaaggctgtggaggggaagcaagacactaaatctgtagataaaaaggatggagctgtgccatctgaggagaagagagacaagaagttggcaaatagcattatcaaattggagaactcgagcccaacaacacaaccaggtgcagatgcgtccgatgttgtatga

SEQ ID NO: 2 - protein, 193 aa, Oryza sativa
MKPDGETQLRPTAAGHPDPGLGTSSAEYVASLGPATAPVSYPYISTYYGGTYGAYSGQPLVNAALMAMPPHSVPLVTDAVVEPIYVNARQYHGILRRRQSRAKAESENKANKIRKPYLHESRHLHALKRARGSGGRFLNSKAVEGKQDTKSVDKKDGAVPSEEKRDKKLANSIIKLENSSPTTQPGADASDVV

SEQ ID NO: 3 - DNA, 20 bp, Artificial Sequence
  primer
atggagtcgaggccgggggg

SEQ ID NO: 4 - DNA, 20 bp, Artificial Sequence
  primer
tcatgtttccttctgtagga

SEQ ID NO: 5 - DNA, 20 bp, Artificial Sequence
  primer
atgaagccagatggtgaaac

SEQ ID NO: 6 - DNA, 20 bp, Artificial Sequence
  primer
tcatacaacatcggacgcat

SEQ ID NO: 7 - DNA, 20 bp, Artificial Sequence
  primer
ggatccctcctgacttacag

SEQ ID NO: 8 - DNA, 20 bp, Artificial Sequence
  primer
ggattcttgctccagaattg

SEQ ID NO: 9 - DNA, 20 bp, Artificial Sequence
  primer
ggagaactcgagcccaacaa

SEQ ID NO: 10 - DNA, 21 bp, Artificial Sequence
  primer
gcaagcacacaattcgatcga

SEQ ID NO: 11 - DNA, 20 bp, Artificial Sequence
  primer
atggagctgctgctgttcta

SEQ ID NO: 12 - DNA, 20 bp, Artificial Sequence
  primer
ttcttccatgctgctctacc